(19) United States
(12) Patent Application Publication
(10) Pub. No.: US 2026/0124460 A1
(43) Pub. Date: May 7, 2026

(54) FIBER OPTIC PROBE FOR THERAPEUTIC USE

(71) Applicant: Rakuten Medical, Inc., San Diego, CA (US)

(72) Inventors: Hideki KANEBAKO, Tokyo (JP); Roland BAYS, Ecublens (CH)

(21) Appl. No.: 19/439,501

(22) Filed: Jan. 5, 2026

Related U.S. Application Data
(63) Continuation of application No. PCT/US2024/037111, filed on Jul. 9, 2024.

(30) Foreign Application Priority Data
Jul. 10, 2023 (JP) 2023-112807

(51) Int. Cl. A61N 5/06; A61K 41/00

(52) U.S. Cl. CPC A61N 5/0603; A61K 41/0042; A61N 5/062; A61N 2005/063; A61N 2005/0659; A61N 2005/0663; A61N 2005/0666

(57) ABSTRACT

The therapeutic fiber optic probe has a light transmission structure 3 capable of outputting light in a predetermined fixed direction, and a mirror structure 4 provided at a position where light output from the light transmission structure can be received, and the mirror structure reflects a portion of the received light required for treatment in a direction different from the fixed direction with a reflectance of 90% or more, and generates a light spot that exhibits a uniform light energy distribution over its entirety.

### CROSS-REFERENCE TO RELATED APPLICATIONS

This application is a Continuation Application of PCT Application No. PCT/US2024/037111, filed Jul. 9, 2024 and based upon and claiming the benefit of priority from prior Japanese Patent Application No. 2023-112807, filed Jul. 10, 2023, the entire contents of which are incorporated herein by reference.

### BACKGROUND OF THE INVENTION

### 1. Field of the Invention

The present invention relates to a fiber optic probe for therapeutic use.

### 2. Description of the Related Art

Currently, there is a treatment method that consists of two steps: administering to a patient a drug containing a conjugate composed of a light-sensitive dye and a component that targets the dye to specific cells, and irradiating the patient with light of a specific wavelength to which the light-sensitive dye reacts. The light-sensitive dye reacts with light in a specific wavelength range. The drug (conjugate) is administered and allowed to selectively target to the specific cells, and then those cells are irradiated with light of a specific wavelength for a certain period of time, activating the light-sensitive dye and causing the specific cells to die or be eliminated through biochemical and physical processes. In delivering the light irradiation to specific tissues, fiber optic probes are used for treatment. For example, phototherapy in which light (including laser light) is irradiated from the surface to affected areas outside the body, or phototherapy in which light (including laser light) is irradiated from the surface to affected areas inside the body, etc. is being performed.

Fiber optic probes used for the above-described phototherapy, are generally forward (front) irradiation type probes (i.e., existing frontal diffusers) that irradiate light from their tips toward the front are used.

In this case, in a frontal diffuser, the direction of light irradiation is limited to only forward (front) from its tip. Therefore, in phototherapy, it is necessary to orient or bend the frontal diffuser so that the tip of the diffuser is directly facing an affected area.

However, the frontal diffuser has a set permissible curvature (bending) radius, and there are certain limitations on the curvature direction and amount. In addition, an irradiation distance from the tip of the frontal diffuser to the irradiated surface must also be kept above a certain distance. Therefore, for example, in a narrow lumen, there may be an area where it is difficult to irradiate light, and in a case where there is an affected part in that area, it may be difficult to smoothly deliver light to perform phototherapy or photoimmunotherapy on that affected part.

To solve this problem, the tip should be configured to irradiate light in a direction other than forward (front), for example, in a sideward (lateral) direction. However, if a typical mirror is used to reflect the light, the reflectance is low. For example, the JIS standard for mirror materials (JIS R 3220:2011) specifies that the reflectance of a mirror should be 83% or higher. In addition, since light-sensitive dyes must be irradiated with a certain amount of light energy in order to be activated by light of a specific wavelength, the light energy must be uniformly irradiated to the irradiated surface. Furthermore, fiber optic probes are also assumed to be used together with endoscopes and catheters. Therefore, the size (diameter) of the fiber optic probe, including the tip, is required to be set to a dimension that allows it to be inserted into an endoscope or catheter.

### BRIEF SUMMARY OF THE INVENTION

One of the purposes of the present invention is to provide a therapeutic fiber optic probe with a tip that is set to dimensions that allow insertion into an endoscope or catheter and that can efficiently irradiate uniform light in a sideward (lateral) direction.

To achieve such a purpose, a therapeutic fiber optic probe according to embodiments comprises a light transmission structure capable of outputting light in a predetermined fixed direction and a mirror structure provided at a position capable of receiving light output from the light transmission structure, in which the mirror structure reflects a portion of received light required for treatment in a direction different from the fixed direction with a reflectance of 90% or more, and generates a light spot that exhibits a uniform light energy distribution over its entirety.

According to the above structure, it is possible to realize a therapeutic fiber optic probe with a tip that is set to dimensions that allow insertion into an endoscope or catheter and that can efficiently irradiate uniform light in a sideward (lateral) direction.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a cross-sectional view of a tip of a therapeutic fiber optic probe according to one embodiment of the present invention.

FIG. 2 is a cross-sectional view of the tip of the therapeutic fiber optic probe viewed from an opening side.

FIG. 3 shows results of evaluation tests for uniformity of reflected light, reflectance, and temperature rise of a mirror structure.

FIG. 4 shows results of evaluation tests for reflectance of specific wavelengths (690 nm and 530 nm bands).

### DETAILED DESCRIPTION OF THE INVENTION

### Description of the Embodiments

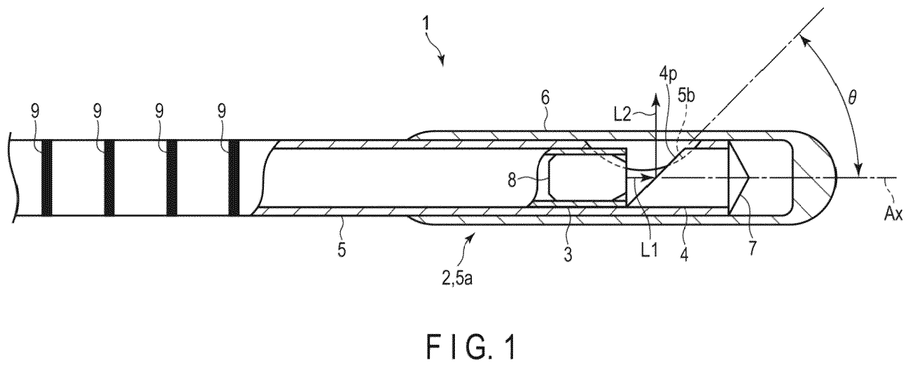

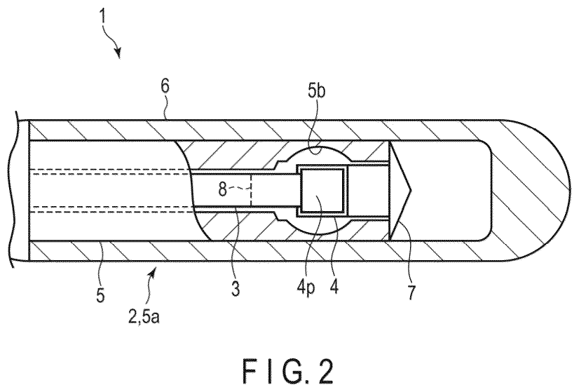

FIG. 1 and FIG. 2 are internal configuration diagrams of a tip 2 of a therapeutic fiber optic probe 1. With the therapeutic fiber optic probe 1, various types of phototherapy (including laser therapy) and/or photoimmunotherapy are performed on an affected area by applying light of a specific wavelength required for treatment by sideward (lateral) irradiation toward the affected area from the tip 2 thereof. Nonlimiting example of such therapies include phototherapy for cancer treatment and for cell and tissue ablation, and photoimmunotherapy, such as for the killing of tumor or tumor cells, premalignant lesions and treatment of cancers.

The therapeutic fiber optic probe 1 is configured to be applicable to both a variation used with an endoscope or catheter (not shown) and a variation not used with an endoscope or catheter (i.e., a variation in which the therapeutic fiber optic probe 1 is used alone).

Therefore, the size (diameter) of the therapeutic fiber optic probe 1, including the tip 2, can be designed to dimensions insertable into an endoscope or catheter. For example, when matching the specifications of a common upper gastrointestinal endoscope, it should be designed to fit into the dimensional size of a forceps opening (working channel) with a diameter of 2.8 mm.

As shown in FIG. 1 and FIG. 2, the therapeutic fiber optic probe 1 includes a light transmission structure 3, a mirror structure 4, a tubular structure 5, a cover structure 6, and a light-shielding structure 7. As the light transmission structure 3, an existing frontal diffuser (i.e., optical fiber) is applied as is, and it is configured to be capable of transmitting various types of light including laser light. A lens structure 8 (objective lens) is mounted at the tip of the light transmission structure 3, and the light transmitted through the light transmission structure 3 is output from the lens structure 8 in a predetermined fixed direction (e.g., forward (front)).

The mirror structure 4 is provided at a position where it can receive the light output from the light transmission structure 3 (hereinafter referred to as output light L1). In this case, the mirror structure 4 is positioned facing the tip of the light transmission structure 3 (lens structure 8). The mirror structure 4 reflects a portion of the received output light L1 that is required for treatment in a direction different from the fixed direction. The light reflected from the mirror structure 4 is hereinafter referred to as reflected light L2. Note that the configuration of the mirror structure 4 is described below.

The light transmission structure 3 and the mirror structure 4 described above are accommodated inside the tubular structure 5. The tubular structure 5 extends to cover the entire length of the light transmission structure 3 and covers the mirror structure 4 so that the positional relationship between the tip of the light transmission structure 3 (lens structure 8) and the mirror structure 4 is maintained constant.

The tubular structure 5 has a metal mesh structure that forms a hollow cylindrical shape. The metal mesh structure is configured, for example, by winding a long, thin wire (not shown) made of metal (e.g., stainless steel) in one direction and also in the opposite direction. In this case, the metal mesh structure is a flexible structure of stainless steel wires wound in alternating directions. This allows the tubular structure 5 to rotate its tip 5a with good followability. As a result, the tip 2 of the therapeutic fiber optic probe 1 can be rotated with good followability as well.

The tip 5a of the tubular structure 5 accommodates the tip of the light transmission structure 3 (lens structure 8) and the mirror structure 4 described above. Thus, the direction of the reflected light L2 from the mirror structure 4, which receives the output light L1 from the light transmission structure 3, is freely adjusted according to the rotation of the tip 5a of the tubular structure 5. As a result, for example, during therapeutic use, the degree of freedom in the irradiation direction or irradiation range of the reflected light L2 from the mirror structure 4 can be increased.

Furthermore, the tubular structure 5 is provided with an opening 5b for passing the reflected light L2 from the mirror structure 4. The cover structure 6 is provided to cover the tip 5a of the tubular structure 5 described above to seal this opening 5b from the outside. Thus, the tip of the light transmission structure 3 (lens structure 8) and the mirror structure 4 described above are accommodated inside the cover structure 6.

In this case, the cover structure 6 is configured by a hollow transparent member (e.g., glass). Thus, the reflected light L2 from the mirror structure 4 passes through the opening 5b of the tubular structure 5, then passes through the cover structure 6 without any excess or deficiency, and the light is emitted directly to the outside.

In some cases, additionally, the tubular structure 5 is provided with a plurality of marks 9 at equal intervals (e.g., 2.5 mm pitch) along its outer circumference. For example, the plurality of marks 9 each form a continuous ring shape along the outer circumference direction of the tubular structure 5 and are fixed by printing or other means. In this case, the thickness and interval pitch of each mark 9 can be set according to the purpose and use of the therapeutic fiber optic probe 1. The plurality of such marks 9 can be used as a positional “ruler” during treatment. For example, in a case of being used with an endoscope, the size and position of an observation target (tissue, affected area) can be visualized using such markings.

Next, the mirror structure 4 described above will be explained in detail.

As shown in FIG. 1 and FIG. 2, the mirror structure 4 comprises a flat reflective portion 4p without any irregularities. The reflective portion 4p is arranged facing the tip of the light transmission structure 3 (lens structure 8). The reflective portion 4p is arranged inclined at a predetermined angle θ and has an upward slope as it moves away from the tip of the light transmission structure 3 (lens structure 8).

Here, the inclination angle θ can be defined as an angle θ formed by the reflective portion 4p with respect to a virtual axis Ax extending parallel along the output light L1 from the light transmission structure 3 (i.e., the angle θ formed between Ax and the reflective portion 4p) as a reference.

In this case, the inclination angle θ can be set according to the purpose or use of the therapeutic fiber optic probe 1. For example, in a case where the reflected light L2 from the mirror structure 4 is inclined 90° with respect to the output light L1 from the light transmission structure 3, the inclination angle θ of the reflective portion 4p should be set to 45°.

Thus, by setting the inclination angle θ of the reflective portion 4p, the reflected light L2 from the mirror structure 4 can be emitted in a direction different from the direction in which the output light L1 from the light transmission structure 3 travels (i.e., in a predetermined fixed direction).

In addition, if the fixed direction is defined as the “forward” direction of the output light L1 from the light transmission structure 3, a different direction is a direction other than the fixed direction, and can be defined as, for example, “sidewards” including a direction that intersects or is orthogonal to the fixed direction. Thus, all of the light that passes through the opening 5b of the tubular structure 5 described below and is emitted outside can be therapeutic light that is output “sidewards” from the tip 2 of the therapeutic fiber optic probe 1.

In embodiments, the reflective portion 4p of the mirror structure 4 has a structure of dielectric multilayer films (not shown) stacked on top of each other. A dielectric multilayer film refers to a reflective mirror having a reflective film in which dielectric thin films with high and low refractive indices are alternately stacked in multiple layers. The reflective portion 4p of the mirror structure 4 is configured in the manner of this reflective mirror.

In some embodiments, additionally, the reflective portion 4p (also called a translucent portion) of the mirror structure 4 is configured to transmit light other than the reflected light L2, which is reflected in a different direction, among the light received. In this case, the mirror structure 4 is provided with the light-shielding structure 7 (e.g., coated with black paint or other manners of blocking light emission) on the opposite side of the reflective portion 4p to shield the transmitted light. This prevents light transmitted through the reflective portion 4p from leaking outside or reaching the reflective portion 4p as returned light.

The light received by the mirror structure 4 (reflective portion 4p) contains multiple wavelengths that differ from each other. The mirror structure 4 (reflective portion 4p) reflects light of all the wavelength, but is designed to have a maximum of reflection for the wavelength of interest. For example, the wavelengths of interest include but are not limited to, light in a wavelength range of 650 to 800 nm can be used for phototherapy or photoimmunotherapy, for example a wavelength of 690 nm, 680 nm, 675 nm, 670 nm or light of a wavelength around or at 530 nm used as guiding light.

In cases where specific wavelengths of light are desired, it is preferable to set the reflectance of the mirror structure 4 (reflective portion 4p) so that at least one wavelength of light having the desired specific wavelength is included among the multiple wavelengths contained in the received light, and so that the reflectance is 90% or more.

Furthermore, as the amount of light reflected from the mirror structure 4 (reflective portion 4p), when the amount of light received by the mirror structure 4 (reflective portion 4p) is 100%, it is preferable that the mirror structure 4 (reflective portion 4p) is set so that the amount of light reflected from the mirror structure 4 (reflective portion 4p), including the amount of light of the specific wavelength, is at least 90% or more.

### Features and Advantages of the Embodiments

According to the present embodiments, the size (diameter) of the therapeutic fiber optic probe 1 including the tip 2 can be set to a dimension that can be inserted into an endoscope or catheter, and therapeutic light can be output from the tip 2 of the therapeutic fiber optic probe 1 sidewards. Furthermore, the tip 2 of the therapeutic fiber optic probe 1 can be rotated with good followability by the tubular structure 5 having a metal mesh structure. Thus, for example, assuming a narrow lumen (e.g., are for treatment), there are areas where it is difficult to irradiate light with a conventional forward-irradiating diffuser, and in a case where there is an affected part in that area, therapy (e.g., phototherapy or photoimmunotherapy) cannot be smoothly performed on that affected part; however, according to the therapeutic fiber optic probe 1 of the present embodiments, the therapeutic light can be effectively irradiated to all affected areas present in a narrow lumen. As a result, the selectivity and effectiveness of the therapy to the affected area is improved.

According to the present embodiments, the inclination angle θ of the reflective portion 4p of the mirror structure 4 can be set according to the purpose and use of the therapeutic fiber optic probe 1. For example, this allows the direction of the therapeutic light output “sidewards” from the tip 2 of the therapeutic fiber optic probe 1 to be set freely.

According to the present embodiments, the tubular structure 5 is provided with a plurality of marks 9 at equal intervals (e.g., 2.5 mm pitch) along its outer circumference. This allows the size and position of the observation target (tissue, affected area) to be visualized, for example, in a case of being used with an endoscope, without having to prepare a measuring tool such as a ruler separately. As a result, the selectivity and effectiveness of therapy for the affected area can be dramatically improved.

In some embodiments, only light of specific wavelengths required for treatment (e.g., 650 to 800 nm, such as 690 nm, wavelength used in phototherapy and photoimmunotherapy and green light in the 530 nm wavelength used as guiding light) is reflected by the reflective portion 4p of the mirror structure 4 and output “sidewards” from the tip 2 of the therapeutic fiber optic probe 1. In this case, the mirror structure 4 (reflective portion 4p) is set so that as the reflectance of the mirror structure 4 (reflective portion 4p), at least one wavelength of light having a specific desired wavelength is included among the multiple wavelengths contained in the received light, and the reflectance is 90% or more and as the amount of light reflected from the mirror structure 4 (reflective portion 4p), when the amount of light received by the mirror structure 4 (reflective portion 4p) is 100%, the amount of light reflected from the mirror structure, including the amount of light of the specific wavelength, is at least 90% or more.

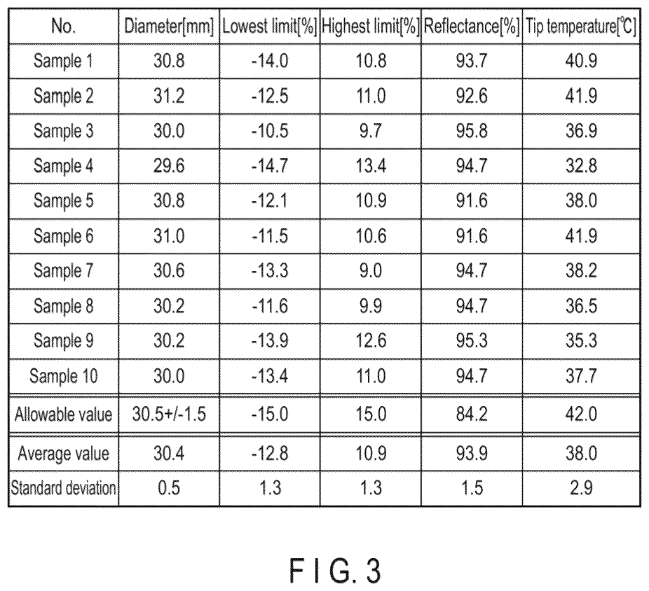

FIG. 3 shows results of an exemplary evaluation test of the therapeutic fiber optic probe 1 having the characteristics and effects described above. In the evaluation test, ten therapeutic fiber optic probes 1 were randomly selected from a plurality of manufactured therapeutic fiber optic probes 1, e.g., as samples 1 to 10, and the irradiation diameter of a light spot generated on a target when the distance between the target and the therapeutic fiber optic probe 1 (specifically, the reflective portion 4p), set to a certain value (e.g., 50 mm), was measured and was confirmed to be in the range of 30.5±1.5 mm.

When the target radiation intensity on a flat irradiated surface was set to 100%, it was confirmed that all radiation intensities within the irradiation circle measured above are in the range of 100%±15% (i.e., in the vicinity of 100%, from −15% to +15%).

From the results of the evaluation test, it was found that although there is the lowest and highest limit of radiation intensity for each sample, both individual samples and the overall average have settled within the range of 100%±15% of the target radiation intensity. At the same time, the reflectance of the reflective portion 4p of the mirror structure 4 was also found to have the characteristics as described above, i.e., light of specific wavelengths (e.g., 690 nm band therapeutic light and 530 nm band guiding light) had a reflectance of 90% or higher. Note that, since these numerical values were measured after being reflected by the mirror structure 4 and transmitted through the cover structure 6, the reflectance at the mirror structure 4 is considered to be slightly larger than the values in FIG. 3. The temperature of the tip 2 of the therapeutic fiber optic probe 1 was measured by starting at a room temperature of 20° C. and then measuring the actual temperature that rise.

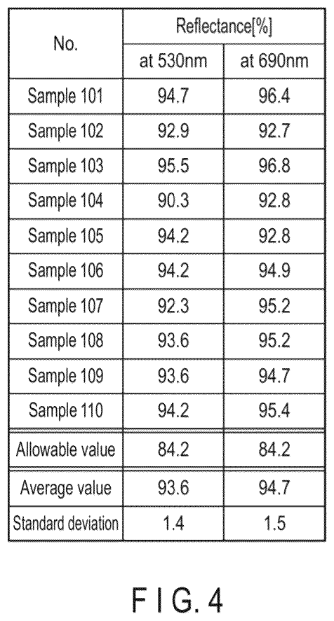

FIG. 4 shows results of an exemplary evaluation test focusing on light of specific wavelengths (690 nm band therapeutic light and 530 nm band guiding light) in ten therapeutic fiber optic probes 1 as new samples 101 to 110, which are different from the above. From the evaluation test results, it was found that the reflectance was almost equal to each other for both types of light (therapeutic light and guiding light). This indicates that the light spot generated by the therapeutic light and the light spot generated by the guiding light have the same size and shape as each other on the target surface, as well as uniform optical characteristics as described below.

By forming a dielectric multilayer film on the reflective portion 4p of the mirror structure 4 (also called microlens) (i.e., by stacking a plurality of dielectric thin films to cover the reflective portion 4p of the mirror structure 4), the light spot as a result of the reflected light L2 being generated on the target (e.g., affected area) surface is a perfect circle and exhibits uniform light energy distribution (i.e., light intensity distribution) over its entirety. As a result, in methods of treating target tissues in subjects described below, such as photodynamic therapy (PDT), photothermal therapy (PTT), photoimmunotherapy (PIT), and other phototherapy methods, a photoactivating compound (e.g., a photoactivatable dye or conjugate) is administered to the subject in advance, which activates a chemical reaction and enables the therapy to be effectively and efficiently applied to the affected area.

In some embodiments, the mutual distance between the therapeutic fiber optic probe 1 (specifically, the reflective portion 4p) and the target (affected area) surface is set to be approximately 5 mm to approximately 80 mm when treating a target tissue in the subject. By arranging and configuring a dielectric multilayer film formed by stacking two or more dielectric thin films on the reflective portion 4p of the mirror structure 4, light of the wavelengths of approximately 660 nm to approximately 820 nm can be optimized; the wavelengths being, for example, approximately 660 nm to approximately 740 nm, or, for example, approximately 660 nm, approximately 670 nm, approximately 675 nm, approximately 677 nm, approximately 680 nm, approximately 685 nm, approximately 690 nm, approximately 695 nm, approximately 700 nm, approximately 705 nm, approximately 710 nm, approximately 715 nm, approximately 720 nm, approximately 725 nm, approximately 730 nm, approximately 735 nm, approximately 740 nm, approximately 745 nm, approximately 750 nm, approximately 755 nm, approximately 760 nm, approximately 765 nm, approximately 770 nm, approximately 775 nm, approximately 780 nm, approximately 785 nm, approximately 790 nm, approximately 795 nm, approximately 800 nm, approximately 805 nm, approximately 810 nm, approximately 815 nm, or approximately 820 nm. In a certain embodiment, light is emitted at two or more wavelengths, such as in the therapeutic light band (e.g., therapeutic light in the 675, 677, 690, 780, or 800 nm band) and in the guiding light band (e.g., guiding light in the 530 nm band). In such examples, only two light rays of specific wavelengths (e.g., therapeutic light in the 675, 677, 690, 780, and 800 nm bands, and guiding light in the 530 nm band) can be reflected with high precision.

In some embodiments of methods of treating a target tissue in a subject, a photoactivating compound (dye or conjugate of a dye) is administered to the subject prior to the application of the light. For example, a photoactivating compound can be a drug. Exemplary photoactivating compounds, include but are not limited to a dye (e.g., phthalocyanine dye, silicon phthalocyanine dye, any dye described in WO2021/207691, or IRDye (registered trademark) 700DX (Rakuten Medical, Inc.)) and such photoactivating compounds conjugated to a targeting agent. In some cases, the targeting agent has specificity for binding to a target on the extracellular surface of cells, such as cancer cells, precancerous cells or immune cells.

Some exemplary targeting agents include antibodies, peptides, or antigen-binding fragments that have the specificity for binding to targets on cancer cells or in the tumor microenvironment. In some cases, the targeting molecule may be or may include a bispecific antibody, scFv, single domain antibody (sdAb) or nanobody, VHH, an isolated single variable domain, affibody, or z-domain structure, DARPin, monobody, anticalin, affilin (registered trademark), affimer type 1 molecule, affimer type 2 molecule, affitin, alphabody, avimer, fynomer, kunitz domain peptide, or nanoclamp. The targeting agents specifically bind to target molecules, e.g., target molecules on the surface of a cell. The target molecules on the cell surface can be extracellular proteins or receptors. Non-limiting examples of the target molecules on the cell surface include an epidermal growth-factor receptor (EGFR), CD25, PD-1, PD-L1, or a prostate-specific membrane antigen (PSMA). In some cases, the targeting agent is an anti-EGFR antibody such as cetuximab.

Exemplary conjugates include, but are not limited to, any conjugate described in U.S. Pat. No. 8,524,239, WO2017031363, and WO2023159182.

In one example of the use of the therapeutic fiber optic probes described in the present specification, the target tissue is a lumen or an opening in the subject, such as esophagus, uterine, vagina, rectum, and colon, and the target tissue is a cancerous tissue, cancerous cells, a tumor, a lesion, or a cancer in an opening in the target (affected area), for example in the esophagus, or endometriosis. In one example, a conjugate of an anti-EGFR antibody (such as cetuximab) and a photoactivatable dye, such as IRDye (registered trademark) 700DX (Rakuten Medical, Inc.), are administered to a subject prior to the application of light at a wavelength of approximately 690 nm using the therapeutic fiber optic probe described herein.

Thus, provided in the present embodiment is a system for treating a subject having a disease or condition, including a light-activatable conjugate, such as any of the conjugates described herein, or a pharmaceutical composition containing any of the conjugates described herein, and a laser capable of emitting light at specific wavelengths. The specific wavelengths are, for example, approximately 650 nm to 820 nm, or approximately 660 nm to 800 nm or approximately 660 nm to approximately 740 nm, or, for example, approximately 660 nm, approximately 670 nm, approximately 675 nm, approximately 677 nm, approximately 680 nm, approximately 685 nm, approximately 690 nm, approximately 695 nm, approximately 700 nm, approximately 705 nm, approximately 710 nm, approximately 715 nm, approximately 720 nm, approximately 725 nm, approximately 730 nm, approximately 735 nm, approximately 740 nm, approximately 745 nm, approximately 750 nm, approximately 755 nm, approximately 760 nm, approximately 765 nm, approximately 770 nm, approximately 775 nm, approximately 780 nm, approximately 785 nm, approximately 790 nm, approximately 795 nm, approximately 800 nm, approximately 805 nm, approximately 810 nm, approximately 815 nm, or approximately 820 nm, and the therapeutic fiber optic probe described herein is operably connected to the laser to deliver the light described above to the target area of the subject. In one example, a system provided for treating a subject with a disease or condition includes a laser capable of emitting light at approximately 675 nm. In one example, a system provided for treating a subject with a disease or condition includes a laser capable of emitting light at approximately 677 nm. In one example, a system provided for treating a subject with a disease or condition includes a laser capable of emitting light at approximately 690 nm. In one example, a system provided for treating a subject with a disease or condition includes a laser capable of emitting light at approximately 780 nm. In one example, a system provided for treating a subject with a disease or condition includes a laser capable of emitting light at approximately 800 nm.

The methods and systems provided herein include methods and systems in a case where the disease or condition to be treated is cancer. In some aspects, the cancer is selected from a group consisting of colon cancer, colorectal cancer, pancreatic cancer, breast cancer, skin cancer, lung cancer, non-small cell lung cancer, renal cell cancer, thyroid cancer, prostate cancer, head and neck cancer, digestive tract cancer, stomach cancer, small bowel cancer, spindle cell tumor, liver cancer, hepatic cancer, bile duct cancer, peripheral nerve cancer, brain cancer, skeletal muscle cancer, smooth muscle cancer, bone cancer, fat tissue cancer, cervix cancer, uterine cancer, genitourinary cancer, lymphoma, and multiple myeloma. In some aspects of the methods of treatment herein, the steps of administering a conjugate and irradiating are repeated. In some aspects, the methods and systems provided herein further include administration of an additional therapeutic agent, such as an additional cancer therapeutic agent (e.g., immunotherapy, radiation therapy, chemotherapy, etc.). In some aspects, the additional therapeutic agent is a checkpoint inhibitor, such as an anti-PD-1 antibody or antigen binding fragment.

In medical applications of photodynamic therapy (PDT) and photoimmunotherapy (PIT), it is required to generate the uniform light spot described above on the target (affected area) surface. Here, in the therapeutic fiber optic probe 1, the output light L1 output from the light transmission structure 3 generally (usually) diverges over a range of 30° to 40° in total angle. For this reason, the reflective portion 4p of the mirror structure 4, which is provided at a position where this output light L1 can be received, has its inclination angle θ set at 45° and is configured by stacking the dielectric multilayer film described above.

The reflective portion 4p on which the dielectric multilayer film is stacked achieves the characteristic of reflecting only light of a specific desired wavelength (e.g., 690 nm band therapeutic light, 530 nm band guiding light) among the received light in the range of 45°±17° (that is, in the vicinity of 45° and in the range of −17° to +17°). This produces the uniform light spot as described herein on the target (affected area) surface.

For example, in a case where the mutual distance between the therapeutic fiber optic probe 1 (specifically, the reflective portion 4p) and the target (affected area of the esophagus) surface is 18 mm, the irradiation diameter of the light spot generated on the target (affected area of the esophagus) surface can be 11 mm, which is sufficient to treat cancerous tissue such as early esophageal cancer.

When a photoactivatable dye or conjugate is present in the target (affected area), a chemical reaction is excited by the application of the specific wavelength of light, resulting in cell killing, thereby reducing or eliminating the lesion (e.g., tumor or tumor cells), and thus treating the disease or condition.

While an embodiment of the present invention has been described, this embodiment has been presented by way of example only, and is not intended to limit the scope of the inventions. The embodiment described herein may be embodied in a variety of other forms; furthermore, various omissions, substitutions and changes in the form of the embodiment described herein may be made without departing from the spirit of the invention. The accompanying claims and their equivalents are intended to cover such embodiment as would fall within the scope and spirit of the inventions.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is

1. A therapeutic fiber optic probe capable of generates a light spot that exhibits a uniform light energy distribution over its entirety comprising:
a light transmission structure;
a lens structure, wherein the lens structure is mounted at the tip of the light transmission structure and is capable of outputting transmitted light in a predetermined fixed direction; and
a mirror structure, wherein the mirror structure comprises a flat reflective portion facing the lens structure, the mirror structure is provided at a position capable of receiving light output from the light transmission structure, wherein the mirror structure reflects a portion of received light in a direction different from the predetermined fixed direction with a reflectance of 90% or more; and
wherein the light transmission structure, the lens and the mirror structure are comprised in a tubular structure, and wherein the tubular structure comprises an opening for passing reflected light from the mirror structure to generate the light spot.

2. The therapeutic fiber optic probe of claim 1, wherein the reflective portion of the mirror structure comprises a dielectric multilayer film in which dielectric thin films having high and low refractive indices are alternately stacked.

3. The therapeutic fiber optic probe of claim 1, wherein the reflective portion of the mirror structure is inclined at a predetermined angle θ.

4. The therapeutic fiber optic probe of claim 3, wherein the predetermined angle is set at 45°.

5. The therapeutic fiber optic probe of claim 1, further comprising a light-shielding structure on the opposite side of the mirror structure to the reflective portion.

6. The therapeutic fiber optic probe of claim 1, further comprising a cover, wherein the cover comprises a hollow transparent member that seals the opening of the tubular structure from the outside.

7. The therapeutic fiber optic probe of claim 6, wherein the cover comprises a glass material.

8. The therapeutic fiber optic probe of claim 6, wherein the lens and the mirror structure are enclosed in a tip of the tubular structure, and wherein the cover encloses the tip of the tubular structure.

9. The therapeutic fiber optic probe of claim 1, wherein the different direction is lateral or sideways as compared to the predetermined fixed direction.

10. The therapeutic fiber optic probe of claim 2, wherein the light received by the mirror structure includes a plurality of wavelengths that differ from each other, and wherein the mirror structure reflects wavelengths of light between 650 nm to 800 nm.

11. The therapeutic fiber optic probe of claim 10, wherein the mirror structure additionally reflects green light in the 530 nm wavelength.

12. A system for treatment comprising the therapeutic fiber optic probe of claim 1 and a photoactivatable compound.

13. A method of treating a subject with a photoactivatable compound and a therapeutic fiber optic probe, wherein the therapeutic fiber optic probe comprises:
a light transmission structure;
a lens structure, wherein the lens structure is mounted at the tip of the light transmission structure and is capable of outputting transmitted light in a predetermined fixed direction; and
a mirror structure, wherein the mirror structure comprises a flat reflective portion facing the lens structure, the mirror structure is provided at a position capable of receiving light output from the light transmission structure, wherein the mirror structure reflects a portion of received light in a direction different from the fixed direction with a reflectance of 90% or more; and
wherein the light transmission structure, the lens and the mirror structure are comprised in a tubular structure, and wherein the tubular structure comprises an opening for passing reflected light from the mirror structure to generate the light spot.

14. The method of claim 13, wherein the photoactivatable compound is a phthalocyanine dye or a conjugate of the phthalocyanine dye, and wherein the photoactivatable compound is administered to a subject prior to administration of light with the therapeutic fiber optic probe.

15. The method of claim 14, wherein the photoactivatable compound comprises a conjugate of IRDye700DX and a targeting agent.

16. The method of claim 15, wherein the subject is treated for a cancer.

17. The method of claim 15, wherein the subject is treated for esophageal cancer, uterus cancer, vaginal cancer, colorectal cancer or endometriosis.

18. A method of producing a light spot of one or more specific wavelengths on a target surface, wherein the method comprises:
transmitting light by a therapeutic fiber optic probe, wherein the therapeutic fiber optic probe comprises:
a light transmission structure;
a lens structure, wherein the lens structure is mounted at the tip of the light transmission structure and is capable of outputting transmitted light in a predetermined fixed direction; and
a mirror structure, wherein the mirror structure comprises a flat reflective portion facing the lens structure, the mirror structure is provided at a position capable of receiving light output from the light transmission structure, wherein the mirror structure reflects a portion of received light in a direction different from the predetermined fixed direction with a reflectance of 90% or more; and
wherein the reflective portion of the mirror structure comprises a dielectric multilayer film in which dielectric thin films having high and low refractive indices are alternately stacked;
wherein the light transmission structure, the lens and the mirror structure are comprised in a tubular structure, and wherein the tubular structure comprises an opening for passing reflected light from the mirror structure to generate the light spot, whereby the light spot exhibits a uniform light energy distribution over its entirety.

19. The method of claim 18, wherein the mirror structure reflects a first wavelength of light between 650 nm to 800 nm.

20. The method of claim 19, wherein the mirror structure additionally reflects a second wavelength of 530 nm, and wherein the light spot generated by the first wavelength and the second wavelength have the same size and shape as each other on the target surface.
